(19) United States
(12) Patent Application Publication
(10) Pub. No.: US 2023/0414812 A1
(43) Pub. Date: Dec. 28, 2023

(54) PORTABLE SANITIZATION DEVICE

(71) Applicant: Sidney Potea Hicks, West Orange, NJ (US)

(72) Inventor: Sidney Potea Hicks, West Orange, NJ (US)

(21) Appl. No.: 17/808,469

(22) Filed: Jun. 23, 2022

(51) Int. Cl. A61L 2/22; A61L 2/24

(52) U.S. Cl. CPC A61L 2/22; A61L 2/24; A61L 2202/14; A61L 2202/15; A61L 2202/16; A61L 2202/17

(57) ABSTRACT

Embodiments of the present invention include a portable device for and method of cleaning and sanitizing surfaces. These surfaces include, but are not limited to, utensils, silverware, gadgets, dishware, and the like. It can be used in a variety of applications, such as commercial use in a kitchen. It is also particularly advantageous in applications where plumbing or a viable water source is not readily available.

### BACKGROUND OF THE INVENTION

The present invention relates to sanitization and, more particularly, to a portable, mechanical, and electrical sanitizer.

Currently, many individuals become ill due to cross-contamination, which can occur at various points in time. For example, cross-contamination can occur when going out to a restaurant, and it can also occur by simply sanitizing cookware and dishes. Allergies, pathogens, and other germs remain a big issue in present-day culture and way of life.

As can be seen, there is a need for a portable device that may be used to clean and sanitize cooking utensils, silverware, gadgets, dishware, and the like. The present invention has many potential applications, including use in commercial kitchens.

### SUMMARY OF THE INVENTION

In one aspect of the present invention, a portable sanitization device is provided that includes: a container configured to be mounted on a body of a user and to retain water therewithin; one or more heating elements disposed in the container and configured to heat the water to a predetermined temperature; a battery electrically coupled to the one or more heating elements; a temperature control electrically coupled between the battery and the one or more heating elements, the temperature control being configured to monitor the temperature of the water and to selectively switch the one or more heating elements on and off based on the monitored temperature; a first hose coupled to the container; and a first nozzle coupled to the first hose and configured to spray the heated water onto a surface.

In another aspect of the present invention, a method of sanitizing a surface is disclosed, with the method comprising the steps of: providing a portable sanitization device comprising: a container; one or more heating elements disposed in the container; a battery electrically coupled to the one or more heating elements; a temperature control electrically coupled between the battery and the one or more heating elements; a first hose coupled to the container; and a first nozzle coupled to the first hose; retaining water in the container; mounting the container on a body of a user; heating the water to a predetermined temperature; monitoring the temperature of the water, using the temperature control; selectively switching the one or more heating elements on and off based on the monitored temperature, using the temperature control; and spraying the heated water onto the surface with the first nozzle.

These and other features, aspects and advantages of the present invention will become better understood with reference to the following drawings, description, and claims.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIG. 1 is a perspective view of an embodiment of the present invention, shown in use;

FIG. 2 is a side elevation view of the embodiment of the present invention;

FIG. 3 is a front elevation view of the embodiment of the present invention; and

FIG. 4 is an electrical diagram of the embodiment of the present invention.

### DETAILED DESCRIPTION OF THE INVENTION

The subject disclosure is described with reference to the drawings, wherein like reference numerals are used to refer to like elements throughout. In the following description, for purposes of explanation, numerous specific details are set forth in order to provide a thorough understanding of the present disclosure such that one skilled in the art will be enabled to make and use the present invention. It may be evident, however, that the present disclosure may be practiced without some of these specific details. For the purpose of clarity, technical material that is known in the technical fields related to the present invention has not been described in detail so that the present invention is not unnecessarily obscured.

Broadly, one embodiment of the present invention is a portable, lightweight cylindrical pump container with two hoses attached to respective nozzles. The pump includes a built-in heating element within the container that heats the water up to a predetermined amount, such as approximately 200-230 degrees Fahrenheit. The water in the container is turned into steam and both water and steam will be used to sanitize, rinse, and clean. The present invention can be used where plumbing and viable water sources are not available.

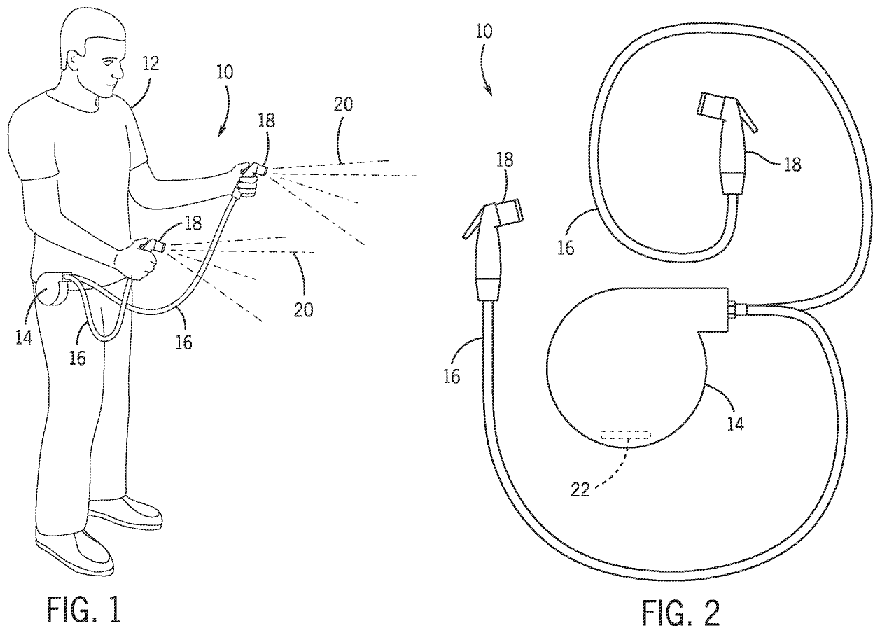

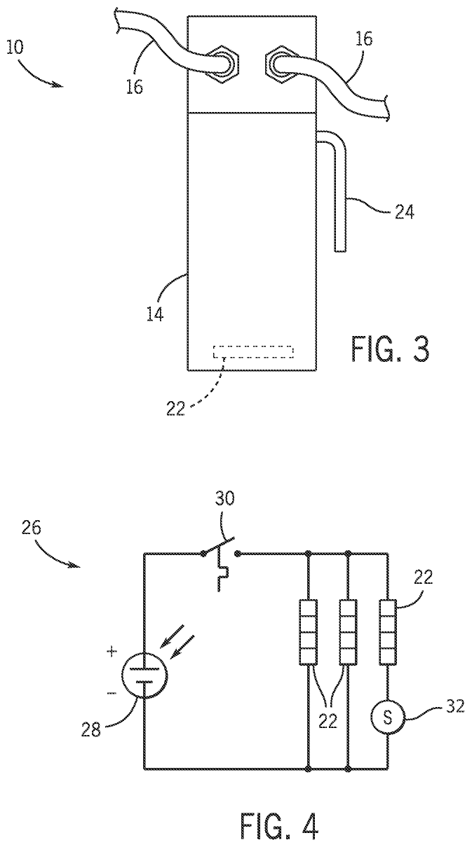

Referring now to FIGS. 1-4, a portable sanitization device 10 that is configured to be body-worn (via a clip 24) or carried by a user 12 is shown. A pump container 14 is formed with a substantially cylindrical shape and includes a heating element 22 therewithin. The heating element 22 is used to heat water 20 within the container 12 (e.g., approximately 2-3 gallons of water 20) to a predetermined temperature range, such as 200-230 degrees Fahrenheit (i.e., approximately a boiling temperature), creating a water and steam combination within the device 10. At least one hose 16, and preferably two, extends from the container 14. The hoses 16 may be formed from heat-resistant plastic in order to prevent degradation of the material over time. Each hose 16 has a nozzle 18 at a distal end thereof which is used as a sprayer. In use, the nozzle 18 sprays a steam and water combination 20 (also referred to herein as heated water).

FIG. 4 illustrates an electrical diagram of operation of the device 10. A battery 28 (such as a solar-charged battery), is electrically coupled to the heating element 22. In embodiments where a solar-charged battery is used, a solar panel may be mounted on an outer-facing surface of the container 14 (such as the main face shown in FIG. 1). Alternatively, the battery 28 may be recharged in other ways, such as via universal serial bus (USB) charging. Electrical energy 32 stored in the battery 28 is used to selectively power the heating element 22. In certain embodiments, the heating element 22 may be a plurality of heating elements 22 operating in parallel. A thermostat 30 is used as a temperature control to regulate the temperature of the steam and water combination 20, such that the one or more heating elements 22 are turned on and off. A switch may also be provided to turn the device 10 on and off manually (e.g., it can be turned off when not being used).

A rise in the temperature of the water 20 within the container 14 results in a corresponding rise in pressure. This pressurizes the liquid/gas combination 20. Thus, when one or more of the nozzles 18 are depressed, a pressurized spray is emitted. This may be a continuous spray. In other embodiments, the nozzle 18 may be embodied in a manner similar to a spray bottle, where a single pump of a trigger results in a short burst of fluid from the container 12 due to suction. Due to the high temperature of the water and steam combination 20, it can be effectively used by the user 12 to sanitize, rinse, and clean a surface and/or any object. In other preferred embodiments (ones that do not rely upon a trigger and a pressurized container to force the water/steam out), a lightweight submersible water pump may be positioned within the container 14 to propel the water and steam combination 20 out of the container 14 at high pressure and high velocity.

Use of the device 10 is simple. A user 12 simply turns it on, clips it to themselves, and wait for the water 20 to heat up to the point it begins to become steam. In certain embodiments, it takes about 4 minutes for the water 20 to heat to that point. Then, one or both of the nozzles 20 are pointed at a location or object to be cleaned, and their trigger(s) depressed. When the location or object no longer needs cleaning, the trigger(s) can be released. As those with skill in the art will appreciate, the present invention may be used in a wide variety of applications, such as cleaning commercial surfaces and/or devices, the sanitization of labs, desks, and other surfaces.

Any appropriate method of manufacturing the present invention may be employed. For example, the electrical heating elements 22 may be formed with embedded sensors or a thermostat 30 may be coupled along the circuit. A battery 28 is integrated along the same circuit. After that, the heating element 22, sensors/thermostat 30, and battery 28 are placed in a formed pump container 14. The container 14 may be formed from, for example, aluminum or another heat-resistant, non-corrosive material. Tube 16 may be formed from a heat-resistant plastic or resin and coupled to the container 14. The nozzles may be commodity items acquired from respective manufacturers, provided that they have the proper specifications to operate in the manner described.

While one or more preferred embodiments are disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

While apparatuses and methods are described in terms of “comprising,” “containing,” or “including” various components or steps, the apparatuses and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A portable sanitization device comprising:
a container configured to be mounted on a body of a user and to retain water therewithin;
one or more heating elements disposed in the container and configured to heat the water to a predetermined temperature;
a battery electrically coupled to the one or more heating elements;
a temperature control electrically coupled between the battery and the one or more heating elements, the temperature control being configured to monitor the temperature of the water and to selectively switch the one or more heating elements on and off based on the monitored temperature;
a first hose coupled to the container; and
a first nozzle coupled to the first hose and configured to spray the heated water onto a surface.

2. The portable sanitization device of claim 1, further comprising a second hose coupled to the container and a second nozzle coupled to the second hose and configured to spray the heated water onto the surface.

3. The portable sanitization device of claim 1, wherein the battery is a solar-charged battery.

4. The portable sanitization device of claim 1, wherein the predetermined temperature is approximately 200 to 230 degrees Fahrenheit.

5. The portable sanitization device of claim 1, further comprising a clip.

6. A method of sanitizing a surface, the method comprising the steps of:
providing a portable sanitization device comprising:
a container;
one or more heating elements disposed in the container;
a battery electrically coupled to the one or more heating elements;
a temperature control electrically coupled between the battery and the one or more heating elements;
a first hose coupled to the container; and
a first nozzle coupled to the first hose;
retaining water in the container;
mounting the container on a body of a user;
heating the water to a predetermined temperature;
monitoring the temperature of the water, using the temperature control;
selectively switching the one or more heating elements on and off based on the monitored temperature, using the temperature control; and
spraying the heated water onto the surface with the first nozzle.
